(19) United States
(12) Patent Application Publication
(10) Pub. No.: US 2004/0258771 A1
(43) Pub. Date: Dec. 23, 2004

(54) Reduced toxicity cisplatin formulations and methods for using the same

(76) Inventor: Patrick Fogarty, Santa Cruz, CA (US)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/803,458

(22) Filed: Mar. 17, 2004

Related U.S. Application Data
(63) Continuation of application No. PCT/US02/29669, filed on Sep. 20, 2002.
(60) Provisional application No. 60/324,566, filed on Sep. 24, 2001.

(51) Int. Cl. A61K 33/24

(52) U.S. Cl. 424/649

(57) ABSTRACT

Methods of using cisplatin active agents in which reduced host toxicity is observed are provided. In the subject methods, an effective amount of a cisplatin active agent is administered to the host in conjunction with the administration of a cisplatin toxicity reducing agent of the present invention. Also provided are compositions for use in practicing the subject methods, e.g., pharmaceutical compositions having reduced toxicity, in which the cisplatin active agent is combined with an cisplatin toxicity reducing agent that reduces the level of undesired cisplatin toxicity while maintaining an effective cisplatin anti-proliferative activity. Also provided are methods of using the subject methods and compositions in the treatment of a variety of different disease conditions.

### CROSS-REFERENCE TO RELATED APPLICATIONS

This application is a continuation of Application serial no. PCT/US02/29669 filed on Sep. 20, 2002; which application, pursuant to 35 U.S.C. § 119 (e), claims priority to the filing date of the U.S. Provisional Patent Application Ser. No. 60/324,566 filed Sep. 24, 2001; the disclosures of which are herein incorporated by reference.

### INTRODUCTION

1. Field of the Invention

The present invention relates to cisplatin and analogues/derivatives thereof.

2. Background of the Invention

Cisplatin—cis-diamine-dichloroplatinum (II)—is one of the more effective anti-tumor agents used in the systemic treatment of germ cell cancers. This chemotherapeutic drug is highly effective in the treatment of tumor models in laboratory animals and in human tumors, such as endometrial, bladder, ovarian and testicular neoplasms, as well as squamous cell carcinoma of the head and neck (Sur, et al., 1983; Steerenberg, et al., 1987).

Like other cancer chemotherapeutic agents, cisplatin is a highly toxic drug. The main disadvantages of cisplatin are its extreme nephrotoxicity, which is the main dose-limiting factor, its rapid excretion via the kidneys, with a circulation half-life of only a few minutes, and its strong affinity to plasma proteins.

Attempts to minimize the toxicity of the drug have included combination chemotherapy, synthesis of cisplatin analogues, immunotherapy and entrapment in liposomes. Antineoplastic agents, including cisplatin, entrapped in liposomes have a reduced toxicity, relative to the agent in free form, while retaining antitumor activity.

However, there is continued interest in the identification of new ways of reducing cisplatin toxicity. The present invention satisfies this need.

Relevant Literature

U.S. Pat. Nos. of interest include: 6,251,355; 6,224,883; 6,130,245; 6,126,966; 6,077,545; 6,074,626; 6,046,044; 6,030,783; 6,001,817; 5,922,689; 4,322,391; and 4,310,515.

### BRIEF SUMMARY OF THE INVENTION

Methods of using cisplatin active agents in which reduced host toxicity is observed are provided. In the subject methods, an effective amount of a cisplatin active agent is administered to the host in conjunction with the administration of a cisplatin toxicity reducing agent of the present invention. Also provided are compositions for use in practicing the subject methods, e.g., cisplatin pharmaceutical compositions having reduced toxicity and kits that include the same. The subject methods and compositions find use in a variety of different applications, including the treatment of a variety of different disease conditions.

### BRIEF DESCRIPTION OF THE FIGURES

FIG. 1 provides a graph of results obtained in an assay measuring tumor growth over time in response to various concentrations of cisplatin and/or TK-211.

### DESCRIPTION OF THE SPECIFIC EMBODIMENTS

Methods of using cisplatin active agents in which reduced host toxicity is observed are provided. In the subject methods, an effective amount of a cisplatin active agent is administered to the host in conjunction with the administration of a cisplatin toxicity reducing agent of the present invention. Also provided are compositions for use in practicing the subject methods, e.g., cisplatin pharmaceutical compositions having reduced toxicity and kits that include the same. The subject methods and compositions find use in a variety of different applications, including the treatment of a variety of different disease conditions.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In addition, many modifications may be made to adapt a particular situation, material, composition of matter, process, process step or steps, to the objective, spirit and scope of the present invention. All such modifications are intended to be within the scope of the claims made herein.

In this specification and the appended claims, the singular forms “a,” “an” and “the” include plural reference unless the context clearly dictates otherwise. Conversely, it is contemplated that the claims may be so-drafted to exclude any optional element. This statement is intended to serve as antecedent basis for use of such exclusive terminology as “solely,” “only” and the like in connection with the recitation of claim elements or by use of a “negative” limitation

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Also, it is contemplated that any optional feature of the inventive variations described herein may be set forth and claimed independently, or in combination with any one or more of the features described herein.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All existing subject matter mentioned herein (e.g., publications, patents, patent applications and hardware) is incorporated by reference herein in its entirety. The referenced items are provided solely for their disclosure prior to the filing date of the present application. Nothing herein is to be construed as an admission that the present invention is not entitled to antedate such material by virtue of prior invention.

In further describing the subject invention, the subject methods are described first in greater detail, followed by a review of the various compositions, e.g., formulations and kits, that may find use in the subject methods, as well as a discussion of various representative applications in which the subject methods and compositions find use.

Methods

As summarized above, methods of administering a cisplatin active agent to a host in need thereof, e.g., for the treatment of a host suffering from disease or condition treatable by a cisplatin active agent (as described in greater detail below), are provided. A feature of the subject methods is that the cisplatin active agent of interest to be administered is administered in conjunction with a cisplatin toxicity reducing agent. By “in conjunction with” is meant that the cisplatin toxicity reducing agent is administered anywhere from simultaneously to up to 5 hours or more, e.g., 10 hours, 15 hours, 20 hours or more, prior to or after the cisplatin active agent. Thus, the toxicity reducing agent and the cisplatin active agent may be administered either: (a) sequentially, with the toxicity reducing agent being administered prior to or after the cisplatin active agent or (b) simultaneously, with the toxicity reducing agent being administered to the subject at the same time as the cisplatin active agent. Where the toxicity reducing agent is administered simultaneously with the cisplatin active agent, the two components may be administered as either a single, combined composition or as two distinct compositions that are simultaneously administered to the host.

In the subject methods, an effective amount of a cisplatin active agent is administered to a host in need thereof in combination with an effective amount of a cisplatin toxicity reducing agent. By cisplatin active agent is meant cisplatin or an analogue/derivative thereof, e.g., native cisplatin and its analogues. Native cisplatin, also referred to herein as cisplatin, is a heavy metal complex containing a central atom of platinum surrounded by two chloride atoms and two ammonia molecules in the cis position. It is a yellow powder with the molecular formula PtCl₂H₆N₂, and a molecular weight of approximately 300 daltons. It is soluble at room temperature in water or saline at 1 mg/ml and has a melting point of 207° C. and decomposes at 270° C. The chlorine atoms in the cisplatin molecule are subject to chemical displacement reactions by nucleophiles, such as water or sulfhydryl groups. In aqueous media, water molecules are potential ligands, which may replace the chlorine atoms to form monohydroxymonochloro cis-diamine platinum (II).

A wide spectrum of cisplatin analogues have been synthesized, offering a different antitumor spectrum, better therapeutic index and reduced toxicity than that offered by native cisplatin. Such analogues include carboplatin, ormaplatin, oxaliplatin, DWA2114R ((-)-(R)-2-aminomethylpyrrolidine (1,1 -cyclobutane dicarboxylato) platinum), zeniplatin, enloplatin, lobaplatin, Cl-973 (SP-4-3(R)-1,1-cyclobutane-dicarboxylato(2-)-(2-methyl-1,4-butanediamine-N, N′)platinum), 254-S nedaplatin and JM-216 (bis-acetato-ammine-dichloro-cyclohexylamine-platinum(IV)) (Weiss, et al., 1993). Some cisplatin analogues, such as spiroplatin, have been found to be more toxic than native cisplatin. While more toxic analogues are not desirable for intravenous administration in free form, such analogues may have use in liposome-entrapped form, which reduces drug toxicity.

Cisplatin active agents of the present invention include cisplatin and any analogues/derivatives thereof whose toxicity is reduced when administered in conjunction with a toxicity reducing agent according to the subject invention. Whether or not a given cisplatin active agent is suitable for use according to the present invention can be readily determined using assays employed in the experimental section, below. Generally, a cisplatin active agent is suitable for use in the subject methods if its toxicity is reduced by at least about 2-fold, usually by at least about 10-fold and more usually by at least about 100-fold, as determined using the Drosophila assay described in the Experimental section, below. In certain embodiments, the cisplatin active agent is one that reduces the occurrence and/or intensity of observable toxic side effects as observed in the mouse assay described in the experimental section below.

By cisplatin toxicity reducing agent is meant an agent that reduces unwanted toxicity of a cisplatin active agent. Toxicity reducing agents of interest are those agents that reduce the toxicity of a cisplatin active agent by at least about 2-fold, usually by at least about 10-fold and more usually by at least about 100-fold, as determined using the Drosophila assay described in the Experimental section, below. In certain embodiments, the toxicity reducing agents of interest are those that reduce the occurrence and/or intensity of observable toxic side effects of a given cisplatin active agent, as observed in the mouse assay described in the experimental section below.

In many embodiments, the toxicity reducing agents of interest are small organic compounds, typically having a molecular mass of from about 100 to about 1,500 daltons. In certain embodiments, the compounds include one or more rings structures, which may or may not be fused and may or may not include one or more heteroatoms, e.g., N, S or O. In certain embodiments, the compounds of interest do not include any ring structures.

Representative toxicity reducing agents include, but are not limited to:

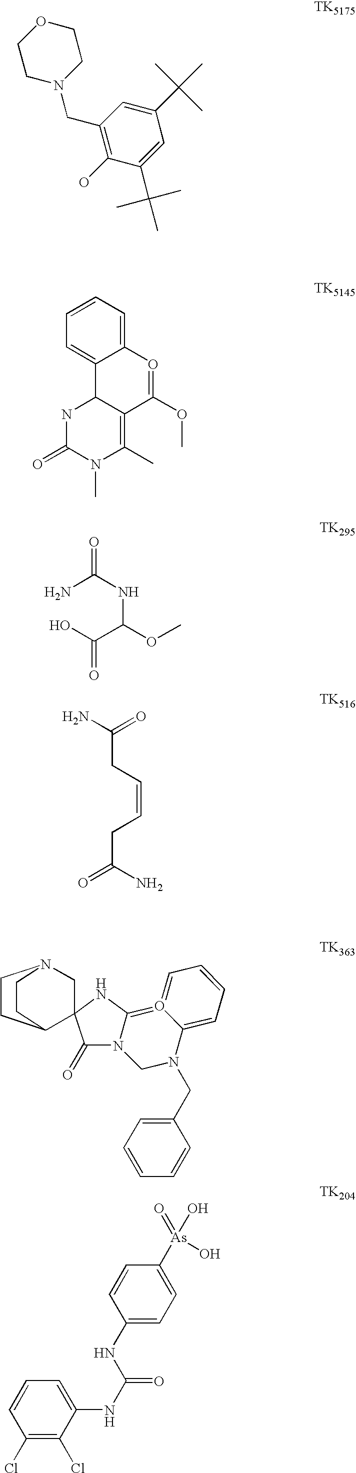

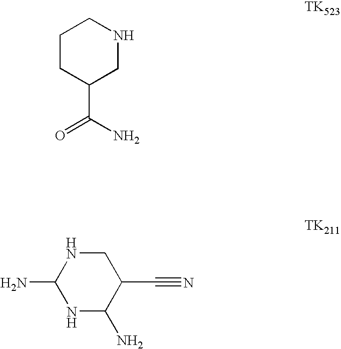

As indicated above, an effective amount of toxicity reducing agent is employed in the subject methods. In certain embodiments, the amount of toxicity reducing agent employed is not more than about the amount of the cisplatin active agent employed. In certain embodiments, an amount is an amount that is less than equimolar to the amount of cisplatin active agent that is administered. Typically, the amount of toxicity reducing agent that is administered is less than about 75%, less than about 50%, less then about 25% and many embodiments less than about 15%, less than about 10% and even less than about 5% or 1% than the amount of cisplatin active agent. In other embodiments, the effective amount is the same as the amount of the active agent, and in certain embodiments the effective amount is an amount that is more than the amount of the cisplatin active agent. Effective amounts can readily be determined empirically using the data provided in the experimental section, below.

Formulations

Also provided are formulations that find use in the practicing the subject invention, where the formulations include at least one of the cisplatin active and the cisplatin toxicity reducing agent in a pharmaceutically acceptable delivery vehicle, such that in certain embodiments, a first formulation of cisplatin active agent and a second formulation of a cisplatin toxicity reducing agent are provided, while in other embodiments a single formulation that includes both the cisplatin active agent and the cisplatin toxicity reducing agent are provided.

In certain embodiments of interest, the cisplatin active agent and the toxicity reducing agent are administered as a single pharmaceutical formulation, that, in addition to including an effective amount of the active agent and toxicity reducing agent, includes other suitable compounds and carriers, and also may be used in combination with other active agents. The present invention, therefore, also includes pharmaceutical compositions comprising pharmaceutically acceptable excipients. The pharmaceutically acceptable excipients include, for example, any suitable vehicles, adjuvants, carriers or diluents, and are readily available to the public. The pharmaceutical compositions of the present invention may further contain other active agents as are well known in the art.

One skilled in the art will appreciate that a variety of suitable methods of administering a formulation of the present invention to a subject or host, e.g., patient, in need thereof, are available, and, although more than one route can be used to administer a particular formulation, a particular route can provide a more immediate and more effective reaction than another route. Pharmaceutically acceptable excipients are also well-known to those who are skilled in the art, and are readily available. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting.

Formulations suitable for oral administration can consist of (a) liquid solutions, such as an effective amount of the compound dissolved in diluents, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The subject formulations of the present invention can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are known in the art to be appropriate.

Suppository formulations are also provided by mixing with a variety of bases such as emulsifying bases or water-soluble bases. Formulations suitable for vaginal administration may be presented as pessaries, tampons, creams, gels, pastes, foams.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Those of skill in the art will readily appreciate that dose levels can vary as a function of the specific compound, the nature of the delivery vehicle, and the like. Preferred dosages for a given compound are readily determinable by those of skill in the art by a variety of means.

The dose administered to an animal, particularly a human, in the context of the present invention should be sufficient to effect a prophylactic or therapeutic response in the animal over a reasonable time frame. One skilled in the art will recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Suitable doses and dosage regimens can be determined by comparisons to anticancer or immunosuppressive agents that are known to effect the desired growth inhibitory or immunosuppressive response. In the treatment of some individuals with the compounds of the present invention, it may be desirable to use a high dose regimen in conjunction with a rescue agent for non-malignant cells. In such treatment, any agent capable of rescue of non-malignant cells can be employed, such as citrovorum factor, folate derivatives, or leucovorin. Such rescue agents are well known to those of ordinary skill in the art. A rescue agent is preferred which does not interfere with the ability of the present inventive compounds to modulate cellular function.

Utility

The subject methods find use in therapeutic applications in which cisplatin administration is indicated. A representative therapeutic application is the treatment of cellular proliferative disease conditions, e.g., cancers and related conditions characterized by abnormal cellular proliferation concomitant. Such disease conditions include cancer/neoplastic diseases and other diseases characterized by the presence of unwanted cellular proliferation, e.g., hyperplasias, and the like.

By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The subject methods find use in, among other applications, the treatment of cellular proliferative disease conditions, including neoplastic disease conditions, i.e., cancers. In such applications, an effective amount of an active agent is administered to the subject in need thereof. Treatment is used broadly as defined above, e.g., to include at least an amelioration in one or more of the symptoms of the disease, as well as a complete cessation thereof, as well as a reversal and/or complete removal of the disease condition, e.g., cure.

There are many disorders associated with a dysregulation of cellular proliferation, i.e., cellular hyperproliferative disorders. The conditions of interest include, but are not limited to, the following conditions.

The subject methods may be employed in the treatment of a variety of conditions where there is proliferation and/or migration of smooth muscle cells, and/or inflammatory cells into the intimal layer of a vessel, resulting in restricted blood flow through that vessel, i.e. neointimal occlusive lesions. Occlusive vascular conditions of interest include atherosclerosis, graft coronary vascular disease after transplantation, vein graft stenosis, peri-anastomatic prosthetic graft stenosis, restenosis after angioplasty or stent placement, and the like.

Diseases where there is hyperproliferation and tissue remodelling or repair of reproductive tissue, e.g. uterine, testicular and ovarian carcinomas, endometriosis, squamous and glandular epithelial carcinomas of the cervix, etc. are reduced in cell number by administration of the subject compounds Tumors of interest for treatment include carcinomas, e.g. colon, duodenal, prostate, breast, melanoma, ductal, hepatic, pancreatic, renal, endometrial, stomach, dysplastic oral mucosa, polyposis, invasive oral cancer, non-small cell lung carcinoma, transitional and squamous cell urinary carcinoma etc.; neurological malignancies, e.g. neuroblastoma, gliomas, etc.; hematological malignancies, e.g. childhood acute leukaemia, acute myelogenous leukemias, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Some cancers of particular interest include breast cancers, which are primarily adenocarcinoma subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas.

Also of interest is non-small cell lung carcinoma. Non-small cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and may vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

Melanoma is a malignant tumor of melanocytes. Although most melanomas arise in the skin, they also may arise from mucosal surfaces or at other sites to which neural crest cells migrate. Melanoma occurs predominantly in adults, and more than half of the cases arise in apparently normal areas of the skin. Prognosis is affected by clinical and histological factors and by anatomic location of the lesion. Thickness and/or level of invasion of the melanoma, mitotic index, tumor infiltrating lymphocytes, and ulceration or bleeding at the primary site affect the prognosis. Clinical staging is based on whether the tumor has spread to regional lymph nodes or distant sites. For disease clinically confined to the primary site, the greater the thickness and depth of local invasion of the melanoma, the higher the chance of lymph node metastases and the worse the prognosis. Melanoma can spread by local extension (through lymphatics) and/or by hematogenous routes to distant sites. Any organ may be involved by metastases, but lungs and liver are common sites.

Other hyperproliferative diseases of interest relate to epidermal hyperproliferation, tissue remodelling and repair. For example, the chronic skin inflammation of psoriasis is associated with hyperplastic epidermal keratinocytes as well as infiltrating mononuclear cells, including CD4+ memory T cells, neutrophils and macrophages.

The methods of the present invention can provide a highly general method of treating many—if not most—malignancies, including tumors derived from cells selected from skin, connective tissue, adipose, breast, lung, stomach, pancreas, ovary, cervix, uterus, kidney, bladder, colon, prostate, central nervous system (CNS), retina and blood, and the like. Representative cancers of interest include, but are not limited to: Head/Neck and Lung tissue (e.g., Head and neck squamous cell carcinoma, Non-small cell lung carcinoma, Small cell lung carcinoma) Gastrointestinal tract and pancreas (e.g., Gastric carcinoma, Colorectal adenoma, Colorectal carcinoma, Pancreatic carcinoma); Hepatic tissue (e.g., Hepatocellular carcinoma), Kidney/urinary tract (e.g., Dysplastic urothelium, Bladder carcinoma, Renal carcinoma, Wilms tumor) Breast (e.g., Breast carcinoma ); Neural tissue (e.g., Retinoblastoma, Oligodendroglioma, Neuroblastoma, Meningioma malignant; Skin (e.g., Normal epidermis, Squamous cell carcinoma, Basal cell carcinoma, Melanoma, etc.); Hematological tissues (e.g., Lymphoma, CML chronic myeloid leukemia, APL acute promyelocytic leukemia, ALL acute lymphoblastic leukemia, acute myeloid leukemia, etc.); and the like.

Particular applications in which the subject methods and compositions find use include those described in U.S. Pat. Nos. 6,251,355; 6,224,883; 6,130,245; 6,126,966; 6,077,545; 6,074,626; 6,046,044; 6,030,783; 6,001,817; 5,922,689; 4,322,391; and 4,310,515; the disclosures of which are herein incorporated by reference.

Kits

Kits with formulations used in the subject methods, are provided. Conveniently, the formulations may be provided in a unit dosage format, which formats are known in the art.

In such kits, in addition to the containers containing the formulation(s), e.g. unit doses, is an informational package insert describing the use of the subject formulations in the methods of the subject invention, i.e. instructions for using the subject unit doses to treat cellular proliferative disease conditions.

These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples further illustrate the present invention and should not be construed as in any way limiting its scope.

### EXPERIMENTAL

I. Lethal Dose (LD) Curve Data

The LD curve in fruit flies was generated for cisplatin. This was achieved by:

Mixing a specific concentration of chemical into the food and water supply of the fruit flies, then 50 wild-type embryos are added to the assay. The LD value for this concentration was calculated by 100−(2×(number of alive flies)). The LD curve was generated by repeating this method over a concentration range. For example, the concentration range tested for cisplatin was 0.01 mM to 100 mM. The LD 98 was identified (for cisplatin this was 5 mM). The LD98 was used as a stringent level for identifying additive chemicals that reduce the toxicity. This stringent level of toxicity is key for several reasons: 1) The high toxicity dose turns even mild toxic side effects into significant barriers for the flies to survive. For example, cisplatin induces toxicity based on heavy metal poisoning and DNA damage. These toxic causes induce different levels of toxic side effects to different target organs and tissues, nephrotoxicity, neurotoxicity, etc. At the LD98 concentration of cisplatin, all of these toxic mechanisms are orders of magnitude above that observed at physiological treatment doses. At the LD98 dose, suppressing any one toxicity side effect will not enable significant survival of the flies. An additive that enables significant survival is more likely able to reduce all toxic side effects of cisplatin.

II. Additive Identification Results

A small molecule library containing 10,000 diverse structures was screened for additive compounds for cisplatin. Fifteen compound additives were found to substantially suppress cisplatin toxicity. TK-211 was one of the compound additives found for cisplatin.

TK-211 is identified as a suppressor of cisplatin induced lethality in the fruit fly

| | % of living flies |
|---|---|
| Fly Assay | (n = 50) |
| Cisplatin (.002 mM) | 94 |
| Cisplatin (.5 mM) | 2 |
| Cisplatin (.5 mM) + TK-211 | 96 |
| (1 uM) | 4 |
| Cisplatin (.5 mM) + Amifostine* | |

*best results from a range of concentrations

Amifostine (Brand name Ethyol) was previously the best and only currently marketed product that reduces the toxicity of cisplatin. The stringency of this invention identifies additives that are dramatically more active in toxicity reduction of cisplatin than any other currently known additive. The stochiometry between parent drug and additive compound (TK-211 above) is key for specificity and not impairing efficacy of parent drug. Toxicity is a gradient, by using the suppression of lethal dose 96% as a screen all unwanted side effects should be suppressed. In addition, compounds that detect survival as few as five flies are detectable.

Other compounds of interest identified along with their fold reduction of cisplatin toxicity in the flies in () were:

TK-295 (225); TK-516 (300); TK-523 (125); TK-363 (80); TK-204 (80); TK-5145 (250); TK-5175 (75).

III. Human Cancer Cell Assessment

Cisplatin has thoroughly demonstrated therapeutic effects in a variety of human cancer cell lines. As a quick secondary screen, the additive alone and in combination with the target drug was examined in these human cancer cell lines. The results of TK-211 are shown as a specific example. The compound alone when treated over a wide concentration range had no effects against the cancer cells. Most importantly, when combined with the target drug, the compound did not alter the anti-cancer activity of the target drug, also over a large range of additive concentrations. This finding is shown below for Ovarian cancer cells, but the activity of cisplatin is unaltered in other human cancer cell types, such as melanoma.

| | conc./test | | |
|---|---|---|---|
| Cpd | (μg/ml) | cancer cell | Cell survival |
| 211 | .02-1.5 | Ovarian | 100% |
| Cis | 2 | Ovarian | 1% |
| Cis | 1 | Ovarian | 3% |
| Cis + TK-211 | 2 + .02 | Ovarian | 1% |

IV. Mouse Testing

The primary aspect is testing in mice for the ability to translate the toxic reducing action of the additive from flies into mice. Cisplatin testing was done using high dose injections of cisplatin or cisplatin/additive mixture.

| Cisplatin alone | cisplatin + TK-211 | TK-211 alone |
|---|---|---|
| LD100 | LD15 | LD0 |
| LD50 | LD0 | N/D |

TK-211 suppresses cisplatin lethality in mice

| | Mouse |
|---|---|
| Mouse Assay | Survival |
| TK-211 (.001-1 mg/kg) | 100% |
| Cisplatin (37 mg/kg) | 0% |
| Cisplatin + TK-211 (37 mg/kg + 0.37 mg/kg) | 100% |
| Cisplatin + Amifostine (37 mg/kg + 200 mg/kg) | 0%* |

*20% of the animals lived 15% longer than controls

The observed effects of the additives identified in flies translate into mice, TK-211 illustrates this above.

TK-211 suppression of lethality in mice translates into suppressing all of cisplatin's unwanted toxic side effects

| | | Cisplatin + | Cisplatin + |
|---|---|---|---|
| Toxic side effect | Cisplatin | TK-211 | Amifostine |
| Weight loss | ++++ | + | +++ |
| Bloody Stool | ++++ | None | ++++ |
| Hypothermia | ++++ | None | +++ |
| Neural Damage | ++++ | None | ++++ |
| Hearing Loss | ++++ | None | ++++ |

Cisplatin side-effects in mice are similar to those observed in patients. As anticipated, additives according to the present invention dramatically reduce all side effects. Amifostine is known only to slightly reduce weight loss and hypothermia.

TK-211 does not alter the efficacy of cisplatin in mice

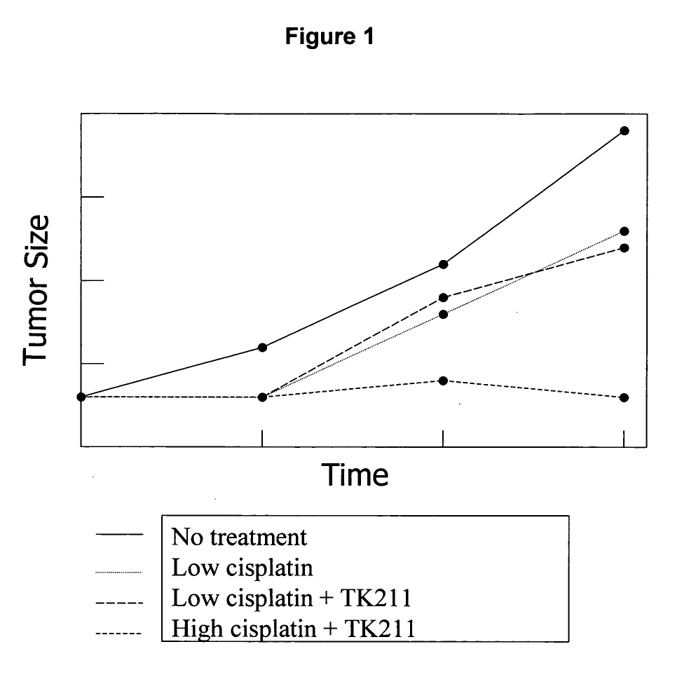

The data shown in FIG. 1 demonstrate that the additives of the present invention do not alter the efficacy of the parent drug. Amifostine has been shown to have a slight impairment of cisplatin efficacy (combined with only slight benefit and the high dose required induces its own side-effects all limit the market potential for this drug). In fact, the additives of the present invention enable higher doses of cisplatin that have significantly beneficial impact, dose levels of cisplatin that are lethal in the absence of the additive.

It is evident from the above results and discussion that the subject invention provides for methods of reducing the unwanted toxicity of cisplatin active agents while retaining their desired activity. As such, the subject invention finds use in a variety of different applications and represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of administering to a subject in need thereof an effective amount of a cisplatin active agent, said method comprising:
administering to said host said effective amount of a cisplatin active agent in conjunction with an amount of a cisplatin toxicity reducing agent effective to reduce toxicity of said cisplatin active agent.

2. The method according to claim 1, wherein said cisplatin active agent and cisplatin toxicity reducing agent are administered at the same time.

3. The method according to claim 2, wherein said cisplatin active agent and cisplatin toxicity reducing agent are administered as separate formulations.

4. The method according to claim 2, wherein said cisplatin active agent and cisplatin toxicity reducing agent are administered in a single formulation.

5. The method according to claim 1, wherein said cisplatin active agent and said cisplatin toxicity reducing agent are administered sequentially.

6. The method according to claim 5, wherein said cisplatin active agent is administered prior to said cisplatin toxicity reducing agent.

7. The method according to claim 5, wherein said cisplatin active agent is administered after said cisplatin toxicity reducing agent.

8. The method according to claim 1, wherein the amount of said cisplatin toxicity reducing agent is not more than about the amount of said cisplatin active agent.

9. The method according to claim 1, wherein said cisplatin active agent is cisplatin.

10. The method according to claim 1, wherein said cisplatin toxicity reducing agent is a small organic compound.

11. The method according to claim 10, wherein said small organic compound is chosen from TK-5175, TK-5145, TK-295, TK-516, TK-363, TK-204, TK-523 and TK-211.

12. A pharmaceutical composition comprising an effective amount of both a cisplatin active agent and an cisplatin toxicity reducing agent in a pharmaceutically acceptable vehicle.

13. The pharmaceutical composition according to claim 12, wherein the amount of said cisplatin toxicity reducing agent is not more than about the amount of said cisplatin active agent.

14. The pharmaceutical composition according to claim 12, wherein said cisplatin active agent is cisplatin.

15. The pharmaceutical composition according to claim 12, wherein said cisplatin toxicity reducing agent is a small organic compound.

16. The pharmaceutical composition according to claim 15, wherein said small organic compound is chosen from TK-5175, TK-5145, TK-295, TK-516, TK-363, TK-204, TK-523 and TK-211.

17. A method of treating a host suffering from a cellular proliferative disease condition, said method comprising:
administering to said host said effective amount of a cisplatin active agent in conjunction with an amount of a cisplatin toxicity reducing agent effective to reduce toxicity of said cisplatin active agent so that said host is treated for said cellular proliferative disease condition.

18. The method according to claim 17, wherein said cisplatin active agent and cisplatin toxicity reducing agent are administered at the same time.

19. The method according to claim 18, wherein said cisplatin active agent and cisplatin toxicity reducing agent are administered as separate formulations.

20. The method according to claim 18, wherein said cisplatin active agent and cisplatin toxicity reducing agent are administered in a single formulation.

21. The method according to claim 17, wherein said cisplatin active agent and said cisplatin toxicity reducing agent are administered sequentially.

22. The method according to claim 21, wherein said cisplatin active agent is administered prior to said cisplatin toxicity reducing agent.

23. The method according to claim 21, wherein said cisplatin active agent is administered after said cisplatin toxicity reducing agent.

24. The method according to claim 17, wherein the amount of said cisplatin toxicity reducing agent is not more than about the amount of said cisplatin active agent.

25. The method according to claim 17, wherein said cisplatin active agent is cisplatin.

26. The method according to claim 17, wherein said cisplatin toxicity reducing agent is a small organic compound.

27. The method according to claim 26, wherein said small organic compound is chosen from TK-5175, TK-5145, TK-295, TK-516, TK-363, TK-204, TK-523 and TK-211.

28. A kit for use in treating a host suffering from a cellular proliferative disease condition, said kit comprising:
(a) a cisplatin active agent; and
(b) a cisplatin toxicity reducing agent.

29. The kit according to claim 28, wherein said cisplatin active agent and cisplatin toxicity reducing agent are present as separate compositions.

30. The kit according to claim 28, wherein said cisplatin active agent and cisplatin toxicity reducing agent are present in the same composition.
